(19) United States
(12) Patent Application Publication
(10) Pub. No.: US 2017/0360732 A1
(43) Pub. Date: Dec. 21, 2017

(54) Methods and Drug Therapies for Patency of Occluded Blood Vessels Following Angioplasty

(71) Applicant: NephroGenesis, LLC, Chattanooga, TN (US)

(72) Inventor: James A. Tumlin, Chattanooga, TN (US)

(21) Appl. No.: 15/533,078

(22) PCT Filed: Dec. 8, 2015
(86) PCT No.: PCT/US15/64519

§ 371 Date: Jun. 5, 2017

Related U.S. Application Data
(60) Provisional application No. 62/088,946, filed on Dec. 8, 2014.

(51) Int. Cl. A61K 31/198; A61K 31/375

(52) U.S. Cl. CPC A61K 31/198; A61K 31/375

(57) ABSTRACT

The present disclosure relates to a method of maintaining luminal patency of a blood vessel following vessel injury, the method including administering a composition comprising at least one lysyl oxidase inhibitor and D-penicillamine to a subject in need thereof Compositions to support the methods are also provided.

### STATEMENT OF PRIORITY

This application claims the benefit, under 35 U.S.C. §119(e), of U.S. Provisional Application Ser. No. 62/088,946 filed Dec. 8, 2014. The entire contents of which are incorporated by reference herein.

### FIELD OF THE INVENTION

The present disclosure relates to methods and therapies designed to maintain the patency of blood vessels, e.g., arteries or veins, that have undergone angioplasty, anthrectomy, surgery and/or any procedure to normalize blood flow through that vessel.

### BACKGROUND

The failure of dialysis accesses remains a leading cause of morbidity and medical costs among end stage renal dialysis (ESRD) subjects. The underlying etiology for dialysis access failure is uniformly due to progressive narrowing of the vessel lumen leading to stasis and thrombosis of the access. The luminal narrowing of arteriovenous fistulae (AVFs) is due to progressive hyperplasia of vessel intima and subsequent infiltration of smooth muscle cells into the vessel media. In an AVF, over 90% of all stenotic lesions are in the vein downstream of the anastomosis. At the histologic level, areas of stenosis within AVFs are characterized by dense neointimal hyperplasia, infiltration of vascular smooth muscle cells and expansion of extracellular matrix material. Moreover, varying types of vascular injury increase the rate of collagen and elastin deposition within the medial and serosal areas of the vessel. The “organization” of neo-collagen and elastin includes the cross-linking of lysine residues leading to increased tensile strength of the fibers. The cross-linking process is catalyzed by the enzyme lysyl oxidase; an extracellular enzyme that oxidatively deaminates lysine residues leading to aldo-condesation and shift-base covalent bonds within and between collagen fibers. The process of cross-linking adjacent collagen fibrils leads to “contraction” tissue and further narrowing of the vessel lumen.

The current standard for restoring blood flow to an obstructed fistula involves the use of percutaneous transluminal angioplasty (PTA) with or without the use of intraluminal stents. While PTA can successfully reduce hyperplastic intima and dilate the vessel media, the very process of angioplasty injures the vessel and contributes to deposition of collagen and elastin fibers. In keeping with these observations, recent studies have shown that repeated angioplasties lead to a progressive loss of vessel lumen diameter over time. This progressive stenosis can lead to re-circulation and loss of fistula function. While the mechanism is incompletely understood, the loss of luminal diameter is due, in part to the cross-linking of collagen and elastin fibers.

Accordingly, what is needed is a method and/or therapy that may be used in conjunction with angioplasty and/or other procedures used to normalize blood flow through a vessel in order to maintain the patency of occluded blood vessels.

### BRIEF SUMMARY

In particular embodiments, the inventive concept relates to a methodology for endovascular administration of a solution of at least two separate drugs that block the activity of lysyl oxidase. It is to be understood that both the foregoing general description and the following detailed description present embodiments of the inventive concept and are intended to provide an overview or framework for understanding the nature and character of the disclosure as it is claimed. Other and further features and advantages of the present inventive concept will be readily apparent to those skilled in the art upon a reading of the following disclosure.

### DETAILED DESCRIPTION

Reference now will be made in detail to the embodiments of the present inventive concept, one or more examples of which are set forth hereinbelow. Each example is provided by way of explanation of the apparatus and/or methods of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as found within the scope of the appended claims and their equivalents. Other objects, features and aspects of the present disclosure are disclosed in or are apparent from the following detailed description. It is to be understood by one of ordinary skill in the art that the present discussion is a description of exemplary embodiments only and is not intended as limiting the broader aspects of the present disclosure.

In particular embodiments, the present inventive concept provides a solution (LysoLox) to be administered endovascularly and directly injected into the walls or arteries or veins that have undergone angioplasty or any other technique designed to restore blood flow through area of stenosis due to atherosclerosis or injury related intimal hyperplasia. This solution (LysoLox) comprises, consists essentially of or consists of one or more direct inhibitors of the enzyme lysyl oxidase and combined with D-penicillamine. This solution will block the following physiologic effects of lysyl oxidase activity in vessel walls after any type of vessel injury: A) lysyl oxidase catalyzed formation of covalent cross-links between individual tropocollagen fibers and blocks the formation of covalent cross-link between adjacent collagen strands in the vessel wall of an artery or vein; B) lysyl oxidase catalyzed formation of covalent cross-links between individual elastin fibers and covalent cross-links between adjacent collagen and elastin fibers in the vessel wall of an artery or vein; C) smooth muscle cell migration into areas of vessel wall injury following angioplasty or any other technique used to restore the blood flow and patency of an occluded artery or vein; and D) Blocks the transcription and synthesis of elastin as well as Type I, Type III and Type IV collagen in the vessel walls of artery or veins that have undergone angioplasty or any procedure designed to restore the blood flow and patency of an occluded artery or vein.

Embodiments of the inventive concept also provide local endovascular administration and direct injection of LysoLox solution into the walls or arteries or veins that have undergone angioplasty or any other technique designed to restore blood flow through area of stenosis due to atherosclerosis or injury related intimal hyperplasia will accomplish the following: A) The inhibition of lysyl oxidase activity will reduce the amount of luminal narrowing due to formation of collagen-collagen and collagen-elastin cross-linkages. This reduction in cross-link formation will reduce the rate of luminal restenosis and in turn reduce the need for serial angioplasties to arteries and veins; and B) The reduction of the need for serial angioplasty or procedures to restore the luminal patency of an artery or vein can be applied to the following clinical conditions: 1) Dialysis arteriovenous fistulae (AVF); 2) Dialysis arteriovenous graft (AVG) with PTFE material or any other material used commercially to create a dialysis AV graft; 3) Central vein stenosis of the Right or Left subclavian veins; 4) Superior Venocava occlusions; 5) Occluded Lower extremity arterial occlusions including but not limited to the common, internal, external iliacs, main and superficial femoral arteries, popliteal artery, anterior tibialis, and any and all arteries that have are amenable to angioplasty or any procedure designed to restore blood flow through that vessel; 6) Native Coronary Arteries, Coronary artery Vein Grafts, Left internal mammary artery grafts used to restore blood flow through coronary arteries with reduced blood flow; and 7) Occluded Left or Right renal arteries.

Embodiments of the inventive concept also provide local delivery of LysoLox solution following initial local injection can be prolonged by the imbedding of various combinations of inhibitors of lysyl oxidase with d-penicillamine into porous “Drug Eluting” stents.

The methods and compositions described herein may comprise, consists essentially of, or consists of the elements described herein.

Hemodialysis, hereinafter referred to as kidney dialysis, or simply “dialysis,” is a medical procedure that is performed on target subjects, for example humans, (and also, on a smaller scale, pet animals), to remove accumulated waste and toxins from the blood in a similar manner to a functioning kidney. When a person or animal's kidneys cease to function properly due to one or more of a number of acute or chronic diseases or conditions (e.g., diabetes, glomerulonephritis and hypertension are commonly recognized medical conditions that are associated with the development of renal failure), toxins accumulate in the bloodstream.

Failure to remove excess water and toxic waste products of routine cellular metabolism—including but not limited to urea, sodium, potassium, phosphate and sulfate as well as nitrogenous waste products such as urea and creatinine leads to the accumulation of these products in the blood and the subsequent condition of uremia; a clinical syndrome that in many aspects resembles systemic poisoning. Ultimately, patients that fail to have these toxins removed by dialysis or other means will die due to cardiac sudden death. Almost every organ system in the body is affected by uremic toxicity and the known uremic clinical symptoms and side effects include, but are not limited to, fatigue, anemia, itching, peripheral neuropathy, gastrointestinal disorders including nausea, vomiting, diarrhea, cardiovascular complications including accelerated coronary and peripheral vascular disease, left ventricular hypertrophy, cardiac fibrosis and accelerated rates of arrhythmias.

Hemodialysis is the clinical process that involved the use of large (for example, 15 gauge) needles to access a arteriovenous fistula (AVF) or arteriovenous graft (AVG) to pump blood through a specialized artificial filter that facilitates the partial removal of water soluble uremic toxins. Each dialysis session typically requires 4 hours and is repeated three per week until either the patient is expires or receives a functional renal transplant. More specifically, dialysis interfaces a flowing stream of blood derived from AVF or AVG with semi-permeable membrane and a rinsing solution called the dialysate. Most dialysate solutions are complex electrolyte solutions that restore necessary electrolytes lost during the dialysis process.

Routine Dialysis Accesses: A typical dialysis access whether it is an AVF or AVG have blood flows that range between 600 and 2000 mls/min. Thus, the accessing of AVFs and AVG three times per week using large hemodialysis needles carries the potential risk for accidental dislodgment of the needles and subsequent massive hemorrhage. As such, hemodialysis is routinely performed in hospitals or free standing clinics that employ specially trained medical personnel.

Dysfunctional Dialysis Accesses: The dysfunctional hemodialysis access remains a significant clinical problem among ESRD subjects, with high primary failure rates and a progressive loss of luminal patency. Failure to maintain the patency of dialysis accesses leads to recurrent hospitalizations and the development of secondary infections^{1,2}. While the AVF is clearly the preferred access for hemodialysis, up to 50% of AVFs never reach maturation, whereas an additional 25% will fail within 2 years³. This failure rate is significantly higher than other elective surgical procedures and has been attributed to numerous reasons, including individual surgical skill and the selection of blood vessels with insufficient caliber and blood flow rates. This observation is illustrated by the higher failure rate of radiocephalic fistulae, which have approximately half the flow rate of brachiocephalic fistulae^{4,5}. Clinically, early failure of a dialysis fistula has been defined as the failure to provide adequate dialysis access for a period of at least 3 months⁶. It is currently estimated that between 23%-46% of all AVFs (both in Europe and the United States) experience early failure or failure to mature, resulting in a 1 year primary patency of only 60%-65%⁷. The failure of fistula maturation is further complicated by the high rates of luminal restenosis leading to even lower secondary patency rates. Both the nephrology and vascular surgery communities have come to rely upon percutaneous transluminal angioplasty (PTA) as a means to maintain dialysis accesses. Balloon angioplasty is a very efficient and highly effective procedure for restoring blood flow to stenotic fistulae. In most cases, the dilation of a stenotic vessel leads to the complete patency without signs of residual stenosis. However, despite this effectiveness, the rate of restenosis following angioplasty remains persistently high at between 36%-62%, with the majority of subjects requiring repeat angioplasty in approximately 3-6 months⁸.

Pathophysiology of Dialysis Access Restenosis: The pathophysiology of venous occlusion in a dialysis access is similar to that observed in atherosclerotic arteries including intimal hyperplasia and smooth muscle cell infiltration. Both the primary and restenotic lesions are composed of hyperplastic neointimal tissues that include advential fibroblasts that migrate into the neointima following mesenchymal transformation into myofibroblasts and smooth muscle cells.

The outer layer of stromal cells in the advential area of the vessel migrates into the intima, thus contributing to the rapid proliferation and expansion. This cell proliferation rate can vary between different vessels, but appears to be accelerated in the veins of diabetic subjects. For example, Chang et al., examined the histology of 10 primary stenotic and 20 restenotic lesions of 30 ESRD subjects with Brescia-Cimino fistulae and measured the number of proliferating cells within the neointima¹¹. When compared to primary stenosis, subjects with restenotic lesions had proportionately higher numbers of proliferating Smooth muscle cells. The level of proliferating smooth muscle cells was inversely related to the interval between angioplasties. The correlation between proliferating smooth muscle cells and fistula survival was also observed between subjects with early and late restenosis. The traditional paradigm for the cellular expansion of the neointima includes the migration of smooth muscle cells from the media to the intima. However, recent data suggest that fibroblasts from the adventia can undergo “mesenchymal transformation” into infiltrating Smooth muscle cells that then populate and expand the intima¹².

Mechanism of Fistula Occlusion: In addition to the above, a growing body of data indicates that increased expression of matrix proteins also contributes to intimal hyperplasia and vessel occlusion. Extracellular matrix (ECM) in both arteries and veins is comprised of structural proteins including collagen and elastin, as well as proteoglycans, glycoproteins, and glycosaminoglycans¹³. While the walls of normal veins have very limited populations of vascular smooth muscle cells and express little elastin, the exposure of veins to arterial pressures following AVF formation can lead to marked increases in both. For example, Abeles et al., examined expression of collagen and elastin fibers in an external jugular model of AVF and found increased collagen deposition was detectable by day 3 whereas elastin was detectable at Day-7¹⁴. These results are very similar to Moczar et al., who found that elastin fibers could be detected within 7 days of fistula creation, but noted that the neo-elastin fibers were abnormal and oriented in the longitudinal axis of the vein as opposed to the typical lamellae pattern¹⁵.

This pathologic remodeling is further enhanced by traumatic injury with balloon angioplasty. Strauss et al. measured the content of collagen and elastin fibers within the arterial walls of rabbit iliac arteries injured by balloon angioplasty and demonstrated that intimal thickness increased 3 fold over a course of 12 weeks. Interestingly, there was minimal cellular proliferation in this model, with only 3.0% of cells undergoing active proliferation. In contrast, the rate of collagen and elastin synthesis increased 4 to 10 fold following angioplasty. This increase in collagen synthesis following vessel wall injury peaks within 48 hours¹⁶. These observations demonstrate that increased synthesis of both collagen and elastin fibers is a principal response to vessel wall injury. Moreover, this rise in collagen production is rapid; peaking within 48 hours of angioplasty. We believe that a strategic administration of inhibitors of collagen and elastin synthesis and cross-linking during this critical first 48 hours will reduce vessel stenosis and resistance to angioplasty.

Lysyl Oxidase is Upregulated in Arterial-Venous Fistulae: In addition to increased expression of ECM proteins, angioplasty and other forms of vessel injury upregulate early response genes involved in tissue repair response including lysyl oxidase. Lysyl oxidase is an amine oxidase expressed and secreted by fibrogenic cells, which facilitate formation of covalent crosslinks between adjacent collagen and elastin fibers. The formation of interfiber crosslinks plays a role in maturation and strengthening of collagen fibers through formation of a shift base covalent bond between lysine side chains. The formation of collagen and elastin cross-links increases the tensile strength of adjoining fibers and is involved in the repair of cardiovascular, respiratory, and skeletal connective tissues. The role of lysyl oxidase in blood vessels is only partially understood, but lysyl oxidase knockout mice are embryonically lethal, while chronic inhibition of lysyl oxidase leads to aneurysms and aortic dissection. Abeles et al., questioned whether the role of lysyl oxidase in collagen and elastin maturation would lead to increased lysyl oxidase expression following vessel injury. Using a mouse model of arteriovenous fistulae, Abeles et al., created a surgical AVF between the carotid artery and external jugular vein and then determined the level of lysyl oxidase expression in the external jugular vein over time. It has been demonstrated that even in the absence of balloon angioplasty, exposure of venous tissues to arterial pressures is sufficient to increase lysyl oxidase expression¹⁴. The time course (48 hrs) of lysyl oxidase expression correlates with the rise in collagen expression noted by Strauss et al., and suggests that increased expression of lysyl oxidase is required to stabilize the comparatively weaker venous tissues¹⁶. This rise in lysyl oxidase expression and its downstream increase in crosslinking of extracellular proteins is thought to contribute to the recoil and luminal narrowing following angioplasty. When collagen and elastin fibers are enzymatically crosslinked by lysyl oxidase, the space between adjacent fibers narrows thereby contributing to “scar contraction”; a phenomenon that is observed in all forms of tissue repair^{17,18}.

Interestingly, recent studies have demonstrated that lysyl oxidase can be transported to the nucleus where its oxidative functions facilitate transcription of specific genes including Type III collagen. This is of particular relevance to the complication of post-angioplasty restenosis in that Type III collagen is the predominant isoform in vascular tissues^{19,20}. Lysyl oxidase may also contribute to post-angioplasty restenosis through its function as a cytokine and chemotactic factor. Recent studies have demonstrated that lysyl oxidase stimulates the chemotaxis and migration of both peripheral blood monocytes as well as vascular smooth muscle cells. For example, Lazarus et al. demonstrated that sub-nanomolar concentrations of lysyl oxidase can stimulate the migration of peripheral blood monocytes by up to 250%, and that this effect could be blocked by β-aminopropionitrile and ethylenediamine; two potent inhibitors of lysyl oxidase activity. In a similar study, Lucero et al. found that lysyl oxidase activity also regulates the migratory rate of vascular smooth muscle cells. β-aminopropionitrile inhibited migration of vascular smooth muscle cells by 80% within 30 minutes^{21,22}. Other inhibitors of lysyl oxidase activity have also been shown to affect cell migration. Using cultured vascular smooth muscle cells, Ivanov et al. studied the effects of ascorbic acid on vascular smooth muscle cell migration and noted that a comparatively low dose of ascorbic acid (100 μM) reduced vascular smooth muscle cell proliferation and migration by up to 16%²³.

Inhibition of Lysyl Oxidase Activity Post Angioplasty: Effect on Vessel Diameter: Previous studies investigated the effect of β-aminopropionitrile on lysyl oxidase activity and its effects on arterial restenosis. β-aminopropionitrile is a lathyrogen and irreversible inhibitor of lysyl oxidase, which subsequently affects the crosslinking of both collagen and elastin fibers. By blocking the lysyl oxidase-induced oxidation of lysine and hydroxylysine residues, β-aminopropionitrile inhibits the formation of immature cross-links in newly synthesized collagen fibers. This early loss of collagen crosslinkage decreases the tensile strength of the fibers and enhances collagenolysis^{24,25}. In a rabbit model of vascular wall injury, Spears et al. induced arterial wall injury using laser radiation or conventional balloon angioplasty, and then compared controls with animals treated with oral β-aminopropionitrile for 6 months. Long-term treatment with β-aminopropionitrile resulted in a 60% reduction in cross-sectional diameter of the arteries injured with balloon angioplasty²⁴. In a similar experiment, Brasselet et al., treated animals in a rabbit model of atherosclerosis with β-aminopropionitrile for 28 days and demonstrated that vessel restenosis following angioplasty was reduced by 33%. β-aminopropionitrile disrupts the normal grouping of collagen fibrils leading to reduced tensile strength. This observation suggests that early restenosis following angioplasty may involve intra- and inter-molecular collagen crosslinking with the potential development of “scar contraction²⁶.

Inhibition of Local Lysyl Oxidase Activity: Role of Ascorbic Acid: Ascorbic acid is an essential cofactor for prolyl hydroxylase, an enzyme that catalyzes the hydroxylation of proline amino acid residues in newly synthesized collagen and elastin fibers. Through its effects on proline hydroxylation, ascorbic acid is necessary for maintaining the triple helix conformation of collagen. Ascorbic acid has been shown to exert a bi-modal effect upon collagen metabolism. At low doses (2 μM-10 μM), ascorbic acid blocks the maturation of collagen fibers by inhibiting lysyl oxidase activity and formation of intra- and inter-collagen crosslinks.

Kuroyanagi et al. demonstrated that 2.0 μM ascorbic acid completely inhibited lysyl oxidase activity within 5 minutes, which then remained 90% inhibited 1 hour after drug administration. Low dose ascorbic acid (0.2 μM) inhibits lysyl oxidase activity by up to 79% (arrow), whereas 10 μM inhibits 100% of lysyl oxidase activity within 15 minutes^{27,35}.

In contrast to its effects on lysyl oxidase, high dose ascorbic acid (100 μM) can stimulate collagen expression. As shown in FIG. 12, 100 μM ascorbic acid increases the expression of Type I and Type IV collagen by 200%-250%. However, this stimulatory effect is limited to Type I and IV collagen. Ascorbic acid has no stimulatory effect upon Type III collagen, which is of significance in that Type III collagen is the predominate isoform in vascular tissue²⁰.

Inhibition of Collagen Cross-Linking with D Penicillamine: D-penicillamine is an α-amino acid metabolite of penicillin that has no antibiotic properties, but is used clinically as a chelating agent, and in the treatment of various autoimmune diseases. In scleroderma, D-penicillamine has been shown to slow fibrosis of the skin and other organs. While its mechanism is not completely understood, its inhibitory effect on collagen metabolism is not due to inhibition of lysyl oxidase, but rather the direct blockade of collagen and elastin crosslink formation. D-penicillamine binds to Allysine residues in collagen fibers leading to the formation of a thiazolidine ring³⁷. This ringed structure sterically blocks the spontaneous aldo condensation of Allysine residues on adjacent collagen strands.

According to some embodiments of the inventive concept, direct endovascular administration of any direct inhibitor of lysyl oxidase in combination with D-penicillamine (LysoLox Solution) will reduce the number of collagen-collagen and collagen-elastin covalent cross-link bonds with the walls of arteries and veins. IN some embodiments, local administration of LysoLox solution to blood vessels that have undergone angioplasty or any procedure that is designed to restore the patency of a blood vessel will slow the rate of restenosis and reduce the need for serial angioplasty.

Endovascular Biopsies of Intimal Tissue: Numerous techniques in both human and animal models have been developed that allow for the safe harvesting of endovascular tissue. For example, Rothman et al. obtained 1.3 mm endovascular biopsies of pulmonary arteries in a dog model of pulmonary hypertension. The tissue demonstrated both medial and intimal hyperplasia. During the procedure, there were no animal deaths, extravasation of contrast material or development of endovascular thrombi. Follow up angiograms 2 and 8 weeks after the biopsy showed widely patent vessels with no evidence of thrombus⁴¹. In a similar manner, Colombo et al. harvested endothelial and smooth muscle cells from arteries and veins from patients with severe heart disease. This procedure was safe and allowed for the comparison of cytokine expression in vascular tissue of normal control and heart failure patients⁴². More recent studies have employed 5 French endovascular forceps to obtain biopsies of endovascular tissue. Morrissey et al. used a similar technique to safely obtain tissue in patients with Takayasu arteritis. The diagnosis of vasculitis was confirmed by the presence of granulomatous changes on the biopsy. Moreover, the use of the forcep allowed for maintenance of tissue orientation, thus allowing the clinicians to monitor changes in intimal hyperplasia following treatment⁴³. Accordingly to some embodiments, in a protocol, 5 French endocardial forcep are used in a manner similar to Morrissey et al. in order to obtain tissue samples from dysfunctional dialysis fistulae. The techniques used by Morrissey et al., and other above techniques have been shown to be safe without the development of bleeding or vessel thrombus. It is important to keep in context that the fistulae that will be studied are active and being actively accessed with 15 gauge needles for 4 hours per session three times per week. Moreover, at the time of the endovascular biopsy, the fistula will have already have been accessed for the purpose of restoring fistula function. Notably, the complete puncture of the fistula at two sites with 15 gauge needles is comparatively more invasive than an endovascular biopsy that does not puncture the vessel.

Intraluminal Administration of Ascorbic Acid and D-Penicillamine: According to some embodiments, local administration of ascorbic acid and D-penicillamine to post-angioplasty areas of a dialysis fistula will reduce collagen deposition and delay maturation thereby prolonging the functional use of the access. The local administration of study drugs allows for maximum delivery to the areas of interest while minimizing toxicities. Because both ascorbic acid and D-penicillamine will be injected directly into the vessel lumen, this approach would be analogous to an intravenous infusion of these drugs. Both ascorbic acid and D-penicillamine have been safely administered intravenously to humans. For example, Stephenson et al., performed a 4 week, Phase I study of high dose intravenous ascorbic acid infusion into 15 oncology patients. The primary objective of this study was to determine whether the anti-oxidant effects of ascorbic acid could enhance the potency of conventional chemotherapy. Patients were given intravenous infusions of ascorbic acid ranging between 70 and 110 gm/M² at a rate of 1000 mg/min. At 70 gms/m2 (approximately 140 gms), the patients achieved a maximum plasma concentration of 50 mM. Despite the rapid infusion of high dose ascorbic acid there were no significant side effects³⁸.

D-penicillamine has also been safely administered intravenously to patients. Wiesner et al. performed a pharmacokinetic study of intravenous D-penicillamine in patients with primary biliary cirrhosis. The half-life following intravenous administration was 86 minutes and did not differ from normal controls. There were no reported side effects with intravenous administration³⁹. In a similar study, Butler et al. dosed patients with 750 mg of oral D-penicillamine and noted a peak plasma level of 3.0 μg/ml within 3 hours⁴⁰. According to some embodiments, 25 μM of D-penicillamine will be injected into the intraluminal space of a dysfunctional dialysis fistula for 2 minutes followed by removal of any unabsorbed solution. This will result in an intraluminal concentration of 3.7 μg/ml, which is consistent with the 3.0 mg/ml plasma levels that are achieved with typical clinical uses of the drug.

According to particular embodiments, the present inventive concept relates generally to an endovascular method of delivering a composition, which may be a solution, of two or more drugs to block the activity (enzymatic, biologic and/or physiological) of lysyl oxidase within the walls of blood vessels. The inhibitory composition including lysyl oxidase (LysoLox solution) is delivered before, during or after angioplasty or any technique that may be used to restore the patency of blood vessels occluded by atherosclerosis, intimal hyperplasia or any occlusive process. Such techniques include, but are not limited to, balloon angioplasty, directional or rotational anthrectomy and/or drug-coated balloons.

In particular embodiments, delivery is post angioplasty delivery. The composition used to inhibit lysyl oxidase activity will include any direct inhibitor of lysyl oxidase or blocker of the substrate for collagen fiber cross-linking and includes but is not limited to the following: B-aminopropionitrile (BAPN), 2-isobutyl-3-chloro- or bromo-allylamine, 2-isopropyl-3-chloro- or bromo-allylamine, 2-sec-butyl-3-chloro- or bromo-allylamine, 2-butyl-3-chloro/bromo-allylamine, 2-hexyl-3-chloro/bromo-allylamine, 2-heptyl-3-chloro/bromo-allylamine, homocysteine, ascorbic acid, poly L-lysine, 2-(9-octadecenyl)-3-chloro- or bromo-allylamine, 2-(3-methyl-3-butenyl)-3-chloro- or bromo-allylamine, lysyl oxidase propeptide, 2-(4-methoxy-2-butenyl)-3-chloro- or bromo-allylamine, 2-thioethoxymethyl-3-chloro- or bromo-allylamine.

Co-administration of a Lysyl Oxidase Inhibitor with D-Penicillamine: The co-administration of an inhibitor of lysyl oxidase with D-Penicillamine may be accomplished by delivery at the same time or sequentially (one before the other in either order of preference for administration) and at the same or different anatomic sites. The compounds by be present in the same formulation or in different formulations. The two may be administered simultaneously (i.e., concurrently) or sequentially. That is, simultaneous administration may be carried out by mixing prior to administration, or by administering at the same point in time but at different anatomic sites or using different routes of administration. Further, the phrases “concurrent administration,” “administration in combination,” “simultaneous administration” or “administered simultaneously” can be employed interchangeably. In some embodiments, ascorbic acid is administered in a range from about 0.2 to 200 μM. In some embodiments, d-penicillamine is administered in a range from about 20 nM-2.0 μM.

Particular routes of administration include parenteral and infusion. Particular parenteral administration routes include intravenous, intraarterial, intramyocardial injection, controlled, delayed release (implantable pumps, or using nanoparticles or other bioengineered materials and/or cells for sustained release.

D-penicillamine is a compound known to be useful in the treatment of diseases and conditions characterized by abnormal collagen deposition such as scleroderma and Idiopathic Pulmonary Fibrosis. Its mechanism of action involves the formation of a thiazolidine ring on deaminated Allysine residues on collagen and elastin fibers. The presence of a thiazolidine ring blocks formation of a Shiff-base covalent bond between Allysine residues formed by the action of Lysyl Oxidase and native lysine residues.

Reduced Dose & Toxicity of Combined inhibitors of Cross-Link Formation: The effective doses of the lysyl oxidase inhibitors that are components of the LysoLox composition or formulation will be expected to be lower when combined with D-Penicillamine. The approach of blocking the formation of collagen-collagen and collagen-elastin cross-links through multiple biochemical pathways offers the potential for reduced toxicity for each component of the LysoLox solution. It is understood that the administration of D-penicillamine will occur with the administration of the LysoLox formula as discussed above.

Real Time Endovascular Delivery of LysoLox Solution: In some embodiments, the LysoLox composition will be administered endovascularly as an injectable drug using any drug delivery catheter that has the ability to occlude the inflow and outflow of artery, vein or vascular graft and a separate chamber that enables delivery of the LysoLox solution under pressure. A combination of inhibitors of lysyl oxidase in varying dosages will be combined with D-penicillamine and administered endovascularly following restoration of the lumen of the occluded artery or vein. The dose of the LO inhibitor will be adjusted to provide the maximum inhibition of lysyl oxidase enzymatic activity and all other physiologically relevant effects of lysyl oxidase activity including but not limited to stimulation of all relevant collagen and elastin isoforms as well as the stimulation of smooth muscle cell migration.

LysoLox Diluent Formulation: The diluent for LysoLox solution will be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,3-dioxolane-4-methanol, ethers such as poly(ethyleneglycol) 400, an oil, a fatty acid, a fatty acid ester or glyceride, or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutically acceptable adjuvants. Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures. The parenteral compositions of this invention will typically contain from about 0.5 to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulations ranges from about 5 to about 15% by weight. The surfactant can be a single component having the above HLB or can be a mixture of two or more components having the desired HLB. Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

Human and Non-Human Subjects for LysoLox Solution: A subject may be a patient. In some embodiments, the subject is a human; however, a subject of this disclosure can include an animal subject, particularly mammalian subjects such as canines, felines, bovines, caprines, equines, ovines, porcines, rodents (e.g. rats and mice), lagomorphs, primates (including non-human primates), etc., including domesticated animals, companion animals and wild animals for veterinary medicine or treatment or pharmaceutical drug development purposes. The subjects relevant to this disclosure may be male or female and may be any species and of any race or ethnicity, including, but not limited to, Caucasian, African-American, African, Asian, Hispanic, Indian, etc., and combined backgrounds. The subjects may be of any age, including newborn, neonate, infant, child, adolescent, adult, and geriatric.

Human Subjects with Normal or Impaired Renal Function: In some embodiments, the subject has impaired kidney function, kidney disease, acute kidney disease, chronic kidney disease or advanced kidney disease. In some embodiments, the subject can have any type of primary disease that leads to impaired glomerular filtration rate (GFR) including but not limited to diabetes, hypertension or glomerulonephritis. In some embodiments, the subject may have normal renal function with occluded arteries or veins in other areas of the vascular anatomy that are not a consequence of reduced renal function. A further subject may be a subject in whom renal dialysis is needed or desired.

As used herein, a “blood vessel” refers to a substantially tubular structure carrying blood through the tissues and organs. Examples include a vein, venule, artery, arteriole or capillary. The blood vessel be obtained from a subject (e.g., a donor, the recipient subject, or a cadaver) or may be a blood vessel engineered using techniques known to those skilled in the art.

As used herein, a “pharmaceutically acceptable carrier” according to the present invention is a component such as a carrier, diluent, or excipient of a composition that is compatible with the other ingredients of the composition in that it can be combined with the agents and/or compositions of the present invention without eliminating the biological activity of the agents or the compositions, and is suitable for use in subjects as provided herein without undue adverse side effects (such as toxicity, irritation, allergic response, and death). Side effects are “undue” when their risk outweighs the benefit provided by the pharmaceutical composition. Non-limiting examples of pharmaceutically acceptable components include, without limitation, any of the standard pharmaceutical carriers such as phosphate buffered saline solutions, water, emulsions such as oil/water emulsions or water/oil emulsions, microemulsions, and various types of wetting agents.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Having now described the invention, the same will be illustrated with reference to certain examples, which are included herein for illustration purposes only, and which are not intended to be limiting of the invention.

### EXAMPLES

### Prophetic Experiments Confirming Efficacy of LysoLox Solution on Restenosis Rates of Arteries or Veins Occluded Hyperplastic Intima

All experiments listed below including without limitation future experiments are intended merely to summarize the assertions made by their authors and no admission is made that any reference constitutes prior art. all papers, publications, patents, patent applications, presentations, texts, reports, manuscripts, brochures, books, internet postings, journal articles, periodicals, and the like, are hereby incorporated by reference into this specification in their entireties.

Study Design: This will be a prospective, randomized, single-blind study examining the safety and efficacy of LysoLox solution (D-penicillamine and ascorbic acid) or any known or potential inhibitor of Lysyl Oxidase activity in patients requiring angioplasty to restore the functional use of a dialysis catheter.

Dysfunctional Dialysis Fistula: Any subject with 1) a venous pressure>250 mmHg for a minimum of 5 minutes at a blood flow of 500 mls/min on two separate dialysis sessions AND at least one of the following (2) a reduction of >0.2 KT/V units over the previous month OR (3) demonstrates evidence of prolonged post-dialysis bleeding will be considered to have a dysfunctional AVF. This definition will be applied to the screening of study subjects as well as the determination of recurrent fistula dysfunction at 12 months.

Subject Screening: Potential subjects will be screened from free standing dialysis centers where the PI or Sub Investigators have stable dialysis patients requiring therapeutic angioplasty of dysfunction dialysis accesses as defined above.

Clinical Experiment #1-Endovascular Biopsy: will identify 30 stable ESRD subjects with pressure-flow or laboratory evidence of dialysis access restenosis (dysfunction), and will be divided into three groups based upon prior duration between serial angioplasties.

Group 1: 10 subjects requiring Low frequency angioplasty

Definition: (0-1 angioplasty during the 12 months prior to randomization)

Group 2: 10 subjects requiring Moderate frequency angioplasty

Definition (2-3 angioplasties during the 12 months prior to randomization)

Group 3: 10 subjects requiring High frequency angioplasty

Definition: (≧4 angioplasties during the 12 months prior to randomization)

Methods: Patients with rising venous or arterial resistance coupled with falling urea clearance (0.2 drop KT/V) will be considered to have “dysfunctional fistula” (See section C4). Patients with “dysfunctional fistulae” will be referred for routine standard of care fistulograms. After giving written informed consent, the degree of luminal narrowing will be determined by the interventional nephrologist. If the fistula lumen is greater than 70% occluded, the patient will undergo intraluminal biopsy of the hyperplastic intima. Samples will be prepared for immunohistochemistry by flash freezing in liquid nitrogen and OCT. A separate biopsy will be fixed in 1% formalin and blocked in paraffin for conventional tissue preparation and staining. A third tissue sample will be used for DNA/RNA analysis, creating primary cultures of endothelial and smooth muscle cells, and enzymatic analysis.

Clinical Experiment #2: Primary Smooth Muscle Cell Cultures:

Primary cultures of vascular smooth muscle cells will be prepared from endoluminal biopsies of hyperplastic intimal tissues. Endovascular tissue will be treated with collagenase (3.0 mg/ml) and elastase (1.0 mg/ml) for 30 minutes at 37° C. The outer adventitia will be mechanically stripped and discarded while the remaining tissue will be further incubated in the collagenase/elastase solution for an additional 1.0 hour at 37° C. The resulting suspension of smooth muscle cells will be centrifuged at 900 rpm for 3 minutes and resuspended in medium 199 and DMEM buffered with 3.7 g/L NaHCO₃ and 5% CO₂, stored at 4° C. Antibiotic supplements will include 100 IU/ml penicillin, 100 mg/ml streptomycin, and 250 ng/ml amphotericin B. Human vascular smooth muscle cells will be serially passaged using 150 cm 2 ml flasks (Corning). When the cultures become confluent, they will be trypsinized (1.25 mg/ml) and subcultured (1:4) for a total of seven passages. No experimental studies will be performed on cells with greater than 7 passages³⁶.

Clinical Experiment #3: Measurement of Lysyl Oxidase Activity: Lysyl oxidase catalyzes the formation of aldehydes from lysine residues in both collagen and elastin precursors. These aldehydes are highly reactive and are essential to the formation of collagen and elastin crosslinks, stabilization of collagen fibrils, and for the integrity and elasticity of mature elastin. Lysyl oxidase activity will be measured in cell homogenates from endovascular biopsies of patients with dysfunctional fistulae using a sensitive lysyl oxidase substrate and detected using a fluorescence microplate reader at Ex/Em=540/590 nm.

Cell suspension from endovascular biopsies will be added 50 U/mL stock solution of Horseradish Peroxidase and then buffered. All reaction assays will be away from direct light and fluorescence determined using plate reader at Ex/Em=540/590 nm and clear bottom 96 well plates. Absorbance at the wavelength of 576±5 nm may be used for specific tissue types.

Clinical Experiment #4: Immunohistochemistry: Tissue obtained by endovascular biopsy will be “flash frozen” at the beside and embedded in OCT or paraffin.

Anticipated Outcome-Clinical Experiments 1-4: We anticipate that compared to Group 1, endovascular tissue samples obtained from Groups 2 and 3 will have higher expression and activity of lysyl oxidase. We further anticipate that patients with the shortest interval between angioplasties (Groups 2 and 3) will have the highest levels of Types I, III, and IV collagen deposition. We anticipate that repeat endovascular biopsies obtained from patients randomized to receive post-angioplasty ascorbic acid and D-penicillamine (Experimental Groups—See Below) will have reduced expression of lysyl oxidase and decreased collagen expression.

Clinical Experiment #5: will identify 30 stable ESRD subjects with pressure-flow or laboratory evidence of dialysis access restenosis (dysfunction), and will be divided into three groups based upon prior duration between serial angioplasties (See below).

Group 1: 10 subjects requiring Low frequency angioplasty

Definition: (0-1 angioplasty during the 12 months prior to randomization)

Group 2: 10 subjects requiring Moderate frequency angioplasty

Definition (2-3 angioplasties during the 12 months prior to randomization)

Group 3: 10 subjects requiring High frequency angioplasty

Definition: (≧4 angioplasties during the 12 months prior to randomization)

Methods: After obtaining written informed consent, subjects in Groups 1,2, and 3 will undergo block randomization to receive standard of care angioplasty with or without post-angioplasty treatment with LysoLox Solution (D-penicillamine 25 μM and ascorbic acid 10 μM). Thus, 5 patients in each of Groups 1, 2, and 3 will be treated with LysoLox and 5 patients will not. Patient receiving LysoLox treatment will be the Experimental Group while those patients receiving NO post-angioplasty LysoLox will be the Control group. This study will be a randomized, single-blind, prospective study in which the LysoLox solution will be delivered to post-angioplasty tissue using the ACT 8 french Occlusion Perfusion Catheter (OPC). Subjects will be followed for 12 months and monitored for signs of fistula dysfunction. When the primary nephrologist determines that the study patient's fistula has become “dysfunctional” they will be referred for a standard of care fistulogram. The time interval between serial fistulograms will be noted, recorded, and analyzed as a secondary endpoint. Patients referred for a repeat fistulogram and having a luminal narrowing of greater than 70% will undergo a second intimal biopsy.

Anticipated Outcome Clinical Experiment #5: We anticipate that patients treated with the LysoLox solution after routine angioplasty will have longer periods between serial angioplasties. Moreover, we anticipate that subjects receiving LysoLox solution will have greater post-angioplasty luminal diameters.

Experimental Group: Patients randomized to the Experimental group will have the ACT-OCP catheter positioned across post-angioplasty areas of stenosis. A fresh 10.0 ml stock solution of LysoLox (D-penicillamine (25 uM) and ascorbic acid (10.0 uM)) will be prepared for each patient randomized to the Experimental Group. The delivery of LysoLox solution to the occluded area will be accomplished using single use disposable dual balloon drug delivery catheter that will allow for delivery of “LysoLox” solution directly to areas of post-angioplasty occlusion. All experiments will deliver LysoLox solution under a pressure of 6 PSI. A volume of 5.3 ml of LysoLox solution will be directly injected directly into the vessel lumen for 2 minute period. All unabsorbed and excess LysoLox solution will be discarded using standard biohazard disposal methods.

Clinical Experiment #5 Clinical Endpoints: To determine the safety of endovascular delivery of LysoLox solution (D-penicillamine 25 uM and ascorbic acid 10 uM) to dysfunctional dialysis fistulae

Safety endpoints will include the following:
1) Percentage of subjects developing neutropenia, defined as WBC<3000 within 30±3 days of LysoLox administration
2) Percentage of subjects developing thrombocytopenia, defined as an absolute platelet count<50,000 or a 75% reduction from baseline within 30±3 days of LysoLox administration
3) Percentage of subjects developing fever, defined as temperature≧100.5° F. within 30±3 days of LysoLox administration
4) Percentage of subjects that develop a maculopapular or erythematous rash within 30±3 days of LysoLox administration 5) Percentage of subjects developing post-procedure thrombosis within 72±12 hours of drug delivery 6) Percentage of subjects developing new aneurysmal dilations within 12 months of drug delivery

Secondary Objectives Experiment #5:
1) To determine the percentage of subjects requiring repeat angioplasty for fistula dysfunction at 12 months in the Control and Experimental Groups.
2) To determine the duration of time between angioplasties for fistula dysfunction at 12 months in the Control and Experimental Groups.
3) To compare the percentage of fistula stenosis at baseline and at the time of repeat fistulogram between the Experimental and Control Groups.
4) To determine the level of expression and activity of lysyl oxidase in biopsy samples obtained from patients with Low, Moderate, and High rates of restenosis.

Clinical Experiment Inclusion Criteria:
1. Age≧18 and ≦90 years old
2. Receiving stable out-subject hemodialysis for a minimum of 3 months
3. Have a lower arm or upper arm arteriovenous fistula that has been cleared for use by the Vascular surgeon or Interventional Nephrologist
4. Have agreed to participate voluntarily and signed and dated an IRB-approved, Subject Informed Consent form
5. Must have a dysfunctional dialysis fistula that meets the following definition: Any subject with 1) a venous pressure>250 mmHg for a minimum of 5 minutes at a blood flow of 500 mls/min on two separate dialysis session AND at least one of the following:
   (A) A reduction of >0.2 KT/V units over the previous month OR
   (B) Demonstrates evidence of prolonged post-dialysis bleeding will be considered to have a dysfunctional AVF. This definition will be applied to the screening of study subjects as well as the determination of recurrent fistula dysfunction at 6 and 12 months.

Clinical Experiment: Exclusion Criteria
1. Be scheduled for surgical revision of the fistula;
2. Have been in another investigational (non-approved) drug or device study within the previous 30 days
3. Have a known allergy to any component of the investigational product
4. Subjects with a “Hero Endovascular Graft” will be excluded
5. Subjects with a known central vein occlusion will be excluded
6. Subjects with a prior stent for correction of a prior stenosis will be excluded
7. Subjects with more than >3 hemodynamically significant stenosis at one time
8. Subjects who are pregnant will be excluded from the trial (pregnancy test will be performed on subjects of child bearing potential). A urine pregnancy test will be utilized.

Drug Administration Procedures

### Treatments Administered and Identity of Investigational Products

Protocol treatment for patients randomized to the Experimental Group consists of a single post-angioplasty, 2 minute exposure of post angioplasty areas of stenosis in the vein or arterial portions of a dialysis fistula to LysoLox solution. This treatment can be performed to stenotic anastomosis or an area of stenosis in the more proximal portions of the vein. Patients randomized to the Control Group will NOT have insertion of a drug delivery catheter or exposure to LysoLox solution. Envelopes containing random treatment assignments will be opened by the Research Pharmacist and will not be disclosed to the patient or the PI. If the treatment blind needs to be broken, the pharmacist will unblind the PI as applicable and it will be documented accordingly.

Study Procedures-Pre-Op/Baseline Visit (Days −30 to Day 0)

Eligibility for study participation will be assessed by the Principal Investigator, sub-investigator, or their delegates, which include the nurse practitioners assigned to a specific dialysis unit. After signing the informed consent, subjects will undergo a routine pre-operative evaluation. These evaluations are to be carried out anytime up to 30 days prior to, or on the day of the primary angioplasty. These evaluations will include the following:
1) Physical examination;
2) Vital signs: blood pressure (BP), heart rate (HR), and temperature;
3) Standard clinical laboratory tests including hematocrit/hemoglobin, and serum chemistries to include SGOT, SGPT, serum creatinine and BUN;
4) Most recent calculated KT/V determination;
5) The investigator must confirm that the subject meets all the inclusion and exclusion criteria for this study prior to enrollment.
   Note: The above evaluations can be performed at any time during the 30 days prior to the screening fistulogram.

DAY 0—Pre-Angioplasty Randomization

After a subject has been deemed eligible for study entry, the subject will be scheduled for a standard of care fistulogram. The SERRI Research Pharmacist will be notified of a potential patient and informed as to the re-stenosis group the randomization envelope should be selected from; i.e. (Group-1 Low restenosis), (Group-2 Moderate restenosis) or (Groups-3 High restenosis). The pharmacist will select a randomization envelope from one of the three risk groups and determine whether the patient has been randomized to the Experimental Group (Receives Post Angioplasty LysoLox) or Control Group (no therapy after angioplasty). For patients randomized to the Experimental group, a freshly prepared 10 ml volume of LysoLox solution will be transported to the angiography suite prior to the patients scheduled procedure.

DAY 0 Venography and Primary Angioplasty

The Interventional Nephrologist will perform a routine standard of care fistulogram and estimate the degree of fistula stenosis.

Option A: If the fistulogram does NOT show a significant obstructing stenosis defined as ≧70% luminal occlusion OR the subject has more than two significant areas of stenosis, the subject will NOT be considered a candidate for the study. Subsequent decisions regarding the need for angioplasty will be left to the discretion of attending interventional nephrologist.

Note: Subjects that do not receive any interventional procedure WILL NOT be included in the data analysis.

Option B: If the interventional nephrologist DOES finds a significant obstructing lesion, a digital image confirming the lumen is occluded by ≧70% will be taken and added to the source documents. The interventional nephrologist will then proceed with obtaining an endovascular biopsy of the hyperplastic intimal tissue using a 5 f Cordis bioptome (model B-18110) (Monheim, Germany). This endovascular biopsy device is 1.8 mm in diameter when closed and obtains a tissue sample that is 4.5 mm³ in volume.

OCT Fresh Frozen Samples:

Three samples will be obtained and removed from the bioptome as to maintain tissue orientation (assumes luminal surface of the sample will be in the bottom of the bioptome chamber). One biopsy sample will be placed in OCT for immediate freezing at the bedside using liquid nitrogen; this sample will be used for immunohistochemistry. Tissue samples will be placed in OCT solution at 90° to the long axis of the OCT chamber in order to maintain tissue orientation during tissue processing and staining. A second biopsy sample will be placed in 1% formalin for future embedding in paraffin for subsequent Hematoxylin-Eosin and Tri-Chrome staining. A third sample will be placed in RNA protection solution for RNA expression assays³².

RNA-DNA Tissue Processing:

A single tissue sample from the bioptome will be submerged in RNAlater® RNA Stabilization Solution; a solution that stabilizes and protects cellular RNA in intact, unfrozen tissue samples. Using this technique, RNA from tissue secured in RNAlaterφ and stored at −20° C. can be stored indefinitely.

Primary Smooth Muscle Cell Cultures:

The following primary antibodies will be used:

1) TGF-b1 (Y369) (1515Q) (Bioworld Technology, Inc., Louis Park, Minn., USA),

2) TbRI (E161) (15100) (Bioworld Technology, Inc., Louis Park, Minn., USA),

3) Smad2/3 (S2) (15100) (Beijing Biosynthesis Biotechnology Co),

4) Smad4 (L43) (15200) (Bioworld Technology, Inc., Louis Park, Minn., USA),

5) Smad7 (Z8-B): sc-101152 (15100) (Santa Cruz Biotechnology, Inc., Santa Cruz, Calif., USA).

The immunostaining density will be characterized as follows: (background) defined as 0, +0 (weak yellow) as 1, ++(yellow) as 2, and +++(brown) as 3 in the semiquantitative analyses.

DAY 0 Primary Angioplasty:

After harvesting 3 tissue samples, patients that have been randomized to the Experimental Group will undergo primary angioplasty using a conventional balloon at a maximum pressure of 20 ATMS. Each subject will receive a minimum 30 seconds of inflation. This procedure will follow routine angioplasty protocols and be conducted at the discretion of the attending interventional nephrologist.

DAY 0 ACT Occlusion Perfusion Catheter and Study Drug Delivery

The ACT-OPC catheter is an FDA approved drug delivery system that has been shown to safely deliver drugs intravascularly to both arteries and veins. We propose that use of the ACT-OPC catheter will allow for safe delivery of LysoLox solution (D-penicillamine and Ascorbic acid) to post-angioplasty dialysis fistulae.

For patients randomized to the Experimental Group, the interventional nephrologist will place an ACT Occlusion Perfusion Catheter over the area of recent angioplasty. The occlusive balloons will be positioned proximal and distal to the area of angioplasty and then inflated. The drug delivery chamber will then be filled with sterile freshly prepared LysoLox solution and injected into the intra-luminal space around the angioplastied area of stenosis. The drug delivery chamber of the ACT Occlusion Perfusion Catheter will be pressurized to 6 PSI and the LysoLox solution exposed to the intra-luminal area of the fistula for 2 minutes. The residual LysoLox solution in the drug delivery chamber of the ACT catheter will be removed and discarded. The occluding balloons of the drug delivery chamber will be deflated and the ACT Occlusion Perfusion Catheter removed; the usual methods for fistula hemostasis will be used and the subject will be allowed to go home as per standard protocol. The patient's fistula WILL be usable with the next out-patient dialysis session.

1 Month Post Procedure Visit
History will be obtained detailing any events related to morbidity of the access graft and rehospitalization.
Venous pressures for each dialysis session beginning with the first post-angioplasty and averaged over a 30 day period. If at any point the patient meets the definition of “dysfunctional fistula”, they can be referred for a repeat fistulogram at the discretion of the rounding nephrologist.
An average of KT/V for the preceding 3 months will be recorded.
Any serious or adverse events during this period will be recorded and notification sent to the IRB.
This visit will be performed by the rounding nephrologists as standard of care at the routine monthly rounding at the dialysis units.

3 Month Post Procedure Visit
History will be obtained detailing any events related to morbidity of the access graft and rehospitalization.
Venous pressures for each dialysis session beginning with the first post-angioplasty and averaged over a 30 day period. If at any point the patient meets the definition of “dysfunctional fistula”, they can be referred for a repeat fistulogram at the discretion of the rounding nephrologist.
An average of KT/V for the preceding 3 months will be recorded.
Any serious or adverse events during this period will be recorded and notification sent to the IRB.
This visit will be performed by the rounding nephrologists as standard of care at the routine monthly rounding at the dialysis units.

6 Month Post Procedure Visit
History will be obtained detailing any events related to morbidity of the access graft and rehospitalization.
Venous pressures for each dialysis session beginning with the first post-angioplasty and averaged over a 30 day period. If at any point the patient meets the definition of “dysfunctional fistula”, they can be referred for a repeat fistulogram at the discretion of the rounding nephrologist.
An average of KT/V for the preceding 3 months will be recorded.
Any serious or adverse events during this period will be recorded and notification sent to the IRB.
This visit will be performed by the rounding nephrologists as standard of care at the routine monthly rounding at the dialysis units.

12 Month Post Procedure Visit
History will be obtained detailing any events related to morbidity of the access graft and rehospitalization.
Venous pressures for each dialysis session beginning with the first post-angioplasty and averaged over a 30 day period. If at any point the patient meets the definition of “dysfunctional fistula”, they can be referred for a repeat fistulogram at the discretion of the rounding nephrologist
An average of KT/V for the preceding 3 months will be recorded.
Any serious or adverse events during this period will be recorded and notification sent to the IRB.
This visit will be performed by the rounding nephrologists as standard of care at the routine monthly rounding at the dialysis units.

[C.13] Removal of Subjects From Therapy or Assessments

Since the treatment is a one-time event, no subjects will be removed from the study once randomized. Subjects who require a surgical procedure or angioplasty to prevent loss of graft function will be considered to have met the study endpoint.

### REFERENCES

1) Dhingra R K, Young E W, Hulbert-Shearon T E, Leavey S F, Port F K. Type of vascular access and mortality in U.S. hemodialysis subjects. Kidney Int 60: 1443-1451, 2001.
2) Roy-Chaudhury P, Kelly B S, Zhang J, Narayana A, Desai P, Melham M, Duncan H, Heffelfinger S C. Hemodialysis vascular access dysfunction: from pathophysiology to novel therapies. Blood Purif 21: 99-110, 2003
3) Dixon B S. Why don't fistulas mature? Kidney Int 70: 1413-422, 2006.
4) Sivanesan S, How T V, Bakran A. Characterizing flow distributions in AV fistulae for hemodialysis access. Nephrol Dial Transplant 13: 3108-3110, 1998.
5) Dixon BS, Novak L, Fangman J. Hemodialysis vascular access survival: upper-arm native arteriovenous fistula. Am J Kidney Dis 39: 92-101, 2002.
6) Beathard G A, Arnold P, Jackson J, Litchfield T. Aggressive treatment of early fistula failure. Kidney Int 64: 1487-1494, 2003.
7) Allon M, Robbin M L. Increasing arteriovenous fistulas in hemodialysis subjects: problems and solutions. Kidney Int 62: 1109-1124, 2002.
8) Safa A A, Valji K, Roberts A C, Ziegler T W, Hye R J, Oglevie S B. Detection and treatment of dysfunctional hemodialysis access grafts: Effect of a surveillance program on graft patency and the incidence of thrombosis. Radiology 199: 653-657, 1996
9) Roy-Chaudhury P, Kelly B S, Narayama A, et al. Hemodialysis vascular access dysfunction from basic biology to clinical intervention. Adv Ren Replace Ther 9: 74-84, 2002.
10) Roy-Chaudhury P, Spergel, L. M., Besarab, A., Asif, A., Ravani, P. Biology of arteriovenous fistula failure. J Nephrol 20: 150-163, 2007.
11) Chang, C. J., Ko, P. J., Hsu, L. A., Ko, Y. S., Ko, Y. L., Chen, C. F., Huang, C. C., Hsu, T. S., Lee, Y. S., Pang, J. H. Highly increased cell proliferation activity in restenotic hemodialysis vascular access after percutaneous transluminal angioplasty: Implication in prevention of restenosis. Am J Kid Dis 43: 74-84, 2004.
12) Lee. T., and Roy-Chaudhury, P. Advances and new frontiers in the pathophysiology of venous neointimal hyperplasia and dialysis access stenosis. Adv Chron Kid Dis 16: 329-338, 2009.
13) Forrester J S, Fishbein M, Helfant R, Fagin J. A paradigm for restenosis based on cell biology: clues for the development of new preventive therapies. J Am Coll Cardiol 17: 759-769, 1991.
14) Abeles, D., Kwei, S., Stavrakis, G., Zhang, Y., Wang, E. T., Garcia-Cardeña, G. Gene expression changes evoked in a venous segment exposed to arterial flow J Vasc Surg 44: 863-870, 2006.
15) Moczar M, Allard R, Robert L, Loisance D, Derouette S, Cachera J P. Biosynthesis of elastin and other matrix-macromolecules in venous arterial prosthesis. Pathol Biol 24: 37-41, 1976.
16) Strauss, B. H., Chisholm, R. J., Keeley, F. W., Gotlieb, A. I. Logan, R. A., Armstrong, P. W. Extracellular matrix remodeling after balloon angioplasty injury in a rabbit model of restenosis. Circ Res 75: 650-658, 1994.
17) Kagan, H. M., Li, W. Lysyl oxidase: Properties, specificity, and biological roles inside and outside of the cell. J Cell Biochem 88: 660-672, 2003.
18) Remus, E. W., O'Donnell, Jr. R. E., Rafferty, K., Weiss, D., Joseph, G., Csiszar, K., Fong, S. F. T., Taylor, R. W. The role of lysyl oxidase family members in the stabilization of abdominal aortic aneurysms. Am J Physiol Heart Circ. Physiol 303: H1067-H1075, 2012.
19) Li, W., Nellaiappan, K., Strassmaier, T., Graham, L., Thomas, K. M., Kagan, H. M. Localization and activity of lysyl oxidase within nuclei of fibrogenic cells. Proc Natl Acad Sci USA 94: 12817-12822, 1997.
20) Giampuzzi, M., Botti, G., Di Luca, M., Arata, L., Ghiggeri, G., Gusmano, R., Ravazzolo, R., and Di Donato, A. Lysyl oxidase activates the transcription activity of human collagen III promoter: Possible involvement of ku antigen. J Biol Chem 275: 36341-36349, 2000.
21) Lucero, H. A., Ravid, K., Grimsby, J. L., Rich, C. B., DiCamillo, S. J., Maki, J. M., Myllyharju, J., Kagan, H. M. Lysyl oxidase oxidizes cell membrane proteins and enhances the chemotactic response of vascular smooth muscle cells. J Biol Chem 283: 24103-24117, 2008.
22) Lazarus, H. M., Cruikshank, W. W., Narasimhan, N., Kagan, H. M., Center, D. M. Induction of human monocyte motility by lysyl oxidase. Matrix Biol 14: 727-731, 1994.
23) Ivanov, V., Ivanova, S., Roomi, W., Kalinovsky, T., Niedzwiecki, A., Rath, M. Extracellular matrix-mediated control of aortic smooth muscle cell growth and migration by a combination of ascorbic acid, lysine, praline, and catechins. J Cardiovasc Pharmacol 50: 541-547, 2007.
24) Spears, R. J., Zhan, H., Khurana, S., Karvonen, R. L., and Karen M. Reisert, K. M. Modulation by beta-aminopropionitrile of vessel luminal narrowing and structural abnormalities in arterial wall collagen in a rabbit model of conventional balloon angioplasty versus laser balloon angioplasty. J Clin Invest 93: 1543-1553, 1984.
25) Levene, C. I. Lathyrism. In Molecular Pathology of Connective Tissues: R. Perez-Tamayo and M. Rojkind, editors. Marcel Dekker, Inc., New York, 175-228, 1973.
26) Brasselet, C., Durand, E., Addad, F., Haj Zen A. A., Smeets M. B., Laurent-Maquin D., Bouthors S., Bellon G., Dominique de Kleijn D., Godeau G., Garnotel, R., Gogly, B., Antoine Lafont, A. Collagen and elastin cross-linking: a mechanism of constrictive remodeling after arterial injury. Am J Physiol Heart Circ Physiol 289: H2228-H2233, 2005.
27) Kuroyanagi, M., Shimamura, E., Kim, M., Arakawa, N., Fujiwara, Y., Otsuka, M. Effects of L ascorbic acid on lysyl oxidase in the formation of collagen cross-links Biosci Biotechnol Biochem 66: 2077-2082, 2002.
28) Bergethon, P. R., Mogayzel, P. J., Franzblau, C. Effect of the reducing environment on the accumulation of elastin and collagen in cultured smooth-muscle cells Biochem J258: 279-284, 1989.
29) Nimni, M. E. Mechanism of Inhibition of Collagen Crosslinking by Penicillamine, Proc. Royal Soc. Med. Volume 70 Supplement 3, pg 65-72, 1997
30) Nimni, M. E. Deshmukh, K., Gerth, N. Collagen defect induced by penicillamine. Nature: New Biology 240: 221-221, 1972.
31) Saito, M., Marumo, K., Fujii, K., Ishioka, N., Single-column high-performance liquid chromatographic-fluorescence detection of immature, mature, and senescent cross-links of collagen. Anal Biochem 253: 26; 32, 1997.
32) Hiemann, N. E., Wellnhofer, E., Roland Hetzer, R., Rudolf Meyer, R., Small vessel disease after heart transplantation: impact of immunologic and non-immunologic risk factors Transplant Int 18: 908-914, 2005.
33) Hassan, H, A., Soliman, G. M. Morphological changes in the cephalic vein of renal failure subjects before arteriovenous fistula (AVF) creation. Egypt J Histol 31: 419-426, 2008.
34) Ivanov, V., Ivanova, S., Roomi, W., Kalinovsky, T., Niedzwiecki, A., Rath, M. Extracellular matrix-mediated control of aortic smooth muscle cell growth and migration by a combination of ascorbic acid, lysine, proline, and catechins. J Cardiovasc Pharmacol 50: 541-547, 2007.
35) Davidson, J. M., LuVallei, P. A., Zoia, O., Quaglino, D., Giro, M. G. Ascorbate differentially regulates elastin and collagen biosynthesis in vascular smooth muscle cells and skin fibroblasts by pre-translational mechanisms. J Biol Chem 272: 345-352, 1997.
36) Proudfoot, D., Shanahan, C. Human vascular smooth muscle cell culture, Ragai R. Mitry and Robin D. Hughes (eds.), Human Cell Culture Protocols, Methods in Molecular Biology vol. 806, page 251-262, 2013.
37) Siegel, R. C. et. al. Collagen cross-linking: Effect of D-penicillamine on cross-linking in-vitro. J Biol Chem 252: 254-259, 1977.
38) Stephenson, C. M., Levin, R. D., Spector, T., Lis, C. G. Phase I clinical trial to evaluate the safety, tolerability, and pharmacokinetics of high-dose intravenous ascorbic acid in patients with advanced cancer. Cancer Chemother Pharmacol 72: 139-146, 2013.
39) Wiesner R H, Dickson E R, Go V L W, Carlson G L: Pharmacokinetics of intravenous D-penicillamine in normals and primary biliary cirrhosis. Gastroenterology 76: 1270, 1979.
40) Butler, M., Carruthers, G., Harth, M., Freeman, D., Percy, J., Rabenstein, D. Pharmacokinetics of reduced D-Penicillamine in patients with Rheumatoid arthritis. Arthritis and Rheumatism 25: 111-116, 1982.
41) Rothman, A., Mann, D. M., House, M. T., Konopka, R. G., Chiles, P. O., Pedersen, C. A., Wolf, P., Moser, K. M. Transvenous procurement of pulmonary artery smooth muscle and endothelial cells using a novel endoarterial biopsy catheter in a canine model. J Am Coll Cardiol 27: 218-224, 1996.
42) Colombo, P. C., Ashton, A. W., Celaj, S., Talreja, A., Banchs, J. E., Dubois, N. B., Marinaccio, M., Malla, S., Lachmann, J., Ware, J. A., LeJemtel, T. H. Biopsy coupled to quantitative immunofluorescence: A new method to study the human vascular endothelium. J Appl. Physiol 92: 1331-1338, 2002.
43) Morrissey, N. J., Goldman, J., Fallon, J. T., Faries, P. L., Marin, M. L., Hollier, L. H., Endovascular aortic biopsy in the diagnosis of takayasu arteritis J Endovasc Ther 10: 136-140, 2003.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. Therefore, the spirit and scope of the appended claims should not be limited to the description of the versions contained therein.

1. A method of maintaining luminal patency of a blood vessel following vessel injury, the method comprising administering a composition comprising at least one lysyl oxidase inhibitor and D-penicillamine to a subject in need thereof.

2. A method of reducing serial angioplasty or procedures to restore luminal patency in a blood vessel or reducing smooth muscle cell migration into an area of a blood vessel wall following blood vessel injury, the method comprising administering a composition comprising at least one lysyl oxidase inhibitor and D-penicillamine to a subject in need thereof.

3. (canceled)

4. A method of inhibiting cross-linking between collagen and/or elastin fibers, the method comprising administering a composition comprising at least one lysyl oxidase inhibitor and D-penicillamine to a subject in need thereof.

5. The method of claim 1, wherein the lysyl oxidase inhibitor is at least one of B-aminopropionitrile (BAPN), 2-isobutyl-3-chloro- or bromo-allylamine, 2-isopropyl-3-chloro- or bromo-allylamine, 2-sec-butyl-3-chloro- or bromo-allylamine, 2-butyl-3-chloro/bromo-allylamine, 2-hexyl-3-chloro/bromo-allylamine, 2-heptyl-3-chloro/bromo-allylamine, homocysteine, ascorbic acid, poly L-lysine, 2-(9-octadecenyl)-3-chloro- or bromo-allylamine, 2-(3-methyl-3-butenyi)-3-chloro- or bromo-allylamine, lysyl oxidase propeptide, 2-(4-methoxy-2-butenyl)-3 -chloro- or bromo-allylamine, 2-thioethoxymethyl-3-chloro- or bromo-allylamine.

6. The method of claim 1, wherein the at least one lysyl oxidase inhibitor and D-penicillamine are administered at the same time.

7. The method of claim 1, wherein the at least one lysyl oxidase inhibitor and D-penicillamine are administered sequentially.

8. The method of claim 1, wherein the at least one lysyl oxidase inhibitor and D-penicillamine are administered at the same or different anatomical sites.

9. The method of claim 1, wherein the at least one lysyl oxidase inhibitor and D-penicillamine are administered to a subject within 48 hours of a procedure to restore patency of a blood vessel.

10. The method of claim 9, wherein the procedure is angioplasty, anthrectomy and/or drug-coated balloon catheterization.

11. The method of claim 1, wherein the at least one inhibitor of lysyl oxidase is administered directly into a blood vessel wall following angioplasty.

12. The method of claim 1, wherein the method reduces the time and/or frequency of re-stenosis following angioplasty.

13. The method of claim 1, wherein the method reduces stenosis of the lumen of a blood vessel where lysyl oxidase activity and/or collagen and/or elastin cross-linking is considered contributory to occlusion of the blood vessel lumen.

14. The method of claim 1, wherein subjects administered at least one inhibitor of lysyl oxidase experience longer intervals between serial angioplasties compared to control.

15. The method of claim 1, wherein subjects administered at least one inhibitor of lysyl oxidase have greater post-angioplasty luminal diameters.

16. The method of claim 1, wherein the subject has experienced or is at risk of experiencing dialysis of an arteriovenous fistulae, dialysis of an arteriovenous graft, central vein stenosis, superior venocava occlusions, occluded lower extremity arterial occlusion, coronary artery vein grafts, left internal mammary artery grafts, occluded left or light renal arteries, end stage renal disease, angioplasty, anthrectomy, drug-coated balloon catheterization, myocardial infarction, intinial hyperplasia, atherosclerosis, vessel occlusion, vasculitis, and/or fibrosis.

17. A composition comprising at least one lysyl oxidase inhibitor and D-penicillamine in a pharmaceutically acceptable carrier.

18. The composition of claim 17, wherein the at least one lysyl oxidase inhibitor is ascorbic acid.
